(19) United States
(12) Patent Application Publication
(10) Pub. No.: US 2025/0235466 A1
(43) Pub. Date: Jul. 24, 2025

(54) METHODS OF TREATING CANCER

(71) Applicant: ENDOCYTE, INC., West Lafayette, IN (US)

(72) Inventor: Alison A. ARMOUR, Zionsville, IN (US)

(21) Appl. No.: 18/999,436

(22) Filed: Dec. 23, 2024

Related U.S. Application Data
(63) Continuation of application No. 17/047,778, filed on Oct. 15, 2020, now Pat. No. 12,208,102.
(60) Provisional application No. 62/659,016, filed on Apr. 17, 2018.
(60) Provisional application No. 62/670,442, filed on May 11, 2018.

(51) Int. Cl. A61K 31/555; A61K 9/00; A61N 5/10; A61P 35/00

(52) U.S. Cl. CPC A61K 31/555; A61K 9/0019; A61N 5/10; A61P 35/00

(57) ABSTRACT

The invention described herein pertains to drug delivery conjugates for targeted therapy. The invention described herein relates to methods of treating PSMA expressing cancers witha compound of the formula 1. The invention described herein also relates to methods of treating PSMA-expressing cancers with a compound of the formula 1 inpatients where stable disease results after treatment with the compound of the formula 1.

### CROSS-REFERENCE TO RELATED APPLICATIONS

This application claims priority under 35 U.S.C. § 119(e) to U.S. Provisional Application Ser. No. 62/659,016 filed on Apr. 17, 2018 and U.S. Provisional Application Ser. No. 62/670,442 filed on May 11, 2018, the entire disclosures of which are incorporated herein by reference.

### FIELD OF THE INVENTION

The invention described herein pertains to drug delivery conjugates for targeted therapy. The invention described herein relates to methods of treating PSMA expressing cancers with a compound of the formula 1. The invention described herein also relates to methods of treating PSMA-expressing cancers with a compound of the formula 1 in patients where stable disease results after treatment with the compound of the formula 1.

### BACKGROUND

Prostate specific membrane antigen (PSMA) is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in the central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase I (FOLH I) or glutamate carboxypeptidase (GCP II) due to its role in the proximal small intestine where it removes γ-linked glutamate from poly-γ-glutamated folate and α-linked glutamate from peptides and small molecules.

PSMA is named largely due to its higher level of expression on prostate cancer cells; however, its particular function on prostate cancer cells remains unresolved. PSMA is over-expressed in the malignant prostate tissues when compared to other organs in the human body such as kidney, proximal small intestine, and salivary glands. Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

Though the activity of the PSMA on the cell surface of the prostate cells remains under investigation, it has been recognized by the inventors herein that PSMA represents a viable target for the selective and/or specific delivery of biologically active agents, including drug compounds to such prostate cells. One such drug compound is Compound 1

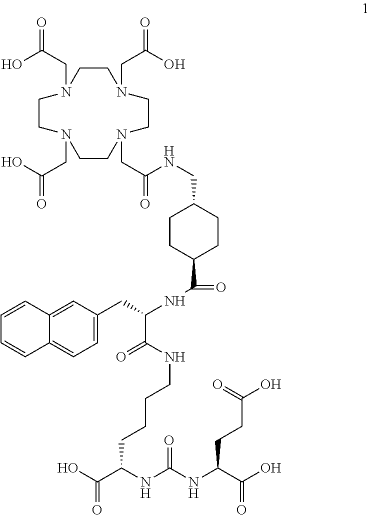 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

(a.k.a. (3S,10S,14S)-3-[(naphthalen-2-yl)methyl]-1,4,12-trioxo-1-[(1R,4S)-4-[[2-[4,7,10-tris(carboxymethyl)-1,4,7,10-tetraazacyclododecan-1-yl]acetamido]methyl]cyclohexyl]-2,5,11,13-tetraazahexadecane-10,14,16-tricarboxylic acid) wherein ¹⁷⁷Lu is complexed to the compound, useful for the treatment of cancer as described in WO2015/055318. Compound 1 can be prepared according to the methods described in WO2015/055318, and WO2015/055318 is incorporated by reference for the preparation of Compound 1, as described in Example 3 and Example 5.

Without being bound by theory, it is believe that PSMA-617 consists of the pharmacophore ligand, glutamate-urea-lysine; the chelator DOTA (able to complex 177Lu); and a linker connecting these 2 entities. It is further believed that the urea-based binding motif allows the agent to bind to, and be internalized by PSMA at the site of disease. It is further believed that the binding of 177Lu-PSMA-617 leads to internalization through endocytosis and a sustained retention of the ligand and its bound radioactive cargo within the cancer cell.

Another such compound is the PSMA-imaging conjugate 4

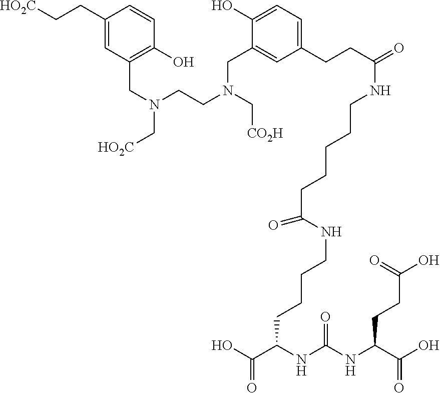 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

(a.k.a. 4,6,12,19-Tetraazadocosane-1,3,7-tricarboxylic acid, 22-[3-[[[2-[[[5-(2-carboxyethyl)-2-hydroxyphenyl]methyl](carboxymethyl) amino]ethyl](carboxymethyl)amino]methyl]-4-hydroxy-phenyl]-5,13,20-trioxo-,(3S,7S)) wherein ⁶⁸Ga (or similar radioactive metal isotope) is complexed to the conjugate, useful for the imaging of cancer as described in Eder M, Schafer M, Bauder-Wust U, Hull W E, Wangler C, Mier W, et al. 68Ga-coiplex lipophilicity and the targeting property of a urea-based PSMA inhibitor for PET imaging. Bioconjug Chem. 2012; 23:688-97. PSMA imaging conjugate 4 can be prepared according to the methods described in (Eder, 2012), and (Eder, 2012) is incorporated by reference for the preparation of PSMA imaging conjugate 4, as described in the examples.

### SUMMARY

In some embodiments, the present disclosure provides a method for treating a cancer in a patient in need of such treatment comprising, administering to the patient a therapeutically effective amount of Compound 1.

In some embodiments, the present disclosure provides use of Compound 1 for treating a cancer in a patient. In some aspects, the use comprises administering to the patient a therapeutically effective amount of the Compound 1.

In some embodiments, the present disclosure provides use of Compound 1 in the preparation of a medicament useful for the treatment of a cancer in a patient. In some aspects, the medicament comprises a therapeutically effective amount of Compound 1.

In some aspects of these embodiments, the cancer is a PSMA expressing cancer. In some aspects of these embodiments, the compound is at least about 98 percent pure. In some embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma.

In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction. In some aspects of these embodiments, the cancer is a primary or secondary brain cancer. In some aspects of these embodiments, the cancer is prostate cancer. In some aspects of these embodiments, the cancer is metastatic prostate cancer.

In some aspects of these embodiments, Compound 1 is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 2 GBq to about 13 GBq. In some aspects of these embodiments, the therapeutically effective amount is from about 4 GBq to about 11 GBq. In some aspects of these embodiments, the therapeutically effective amount is from about 5 GBq to about 10 GBq. In some aspects of these embodiments, the therapeutically effective amount is from about 6 GBq to about 9 GBq. In some aspects of these embodiments, the therapeutically effective amount is from about 6.5 GBq to about 8.5 GBq. In some aspects of these embodiments, the therapeutically effective amount is from about 7 GBq to about 8 GBq. In some aspects of these embodiments, the therapeutically effective amount is about 7.4 GBq. In some aspects of these embodiments, the total dose ranges from about 15 GBq to about 200 GBq. In some aspects of these embodiments, the total dose ranges from about 25 GBq to about 185 GBq. In some aspects of these embodiments, the total dose ranges from about 35 GBq to about 150 GBq. In some aspects of these embodiments, the total dose ranges from about 40 GBq to about 100 GBq. In some aspects of these embodiments, the total dose is about 44 GBq. In some aspects of these embodiments, the maximum duration of treatment of a subject is about 19 to 23 months.

In some aspects of these embodiments, the therapeutically effective amount is from 2 GBq to 13 GBq. In some aspects of these embodiments, the therapeutically effective amount is from 4 GBq to 11 GBq. In some aspects of these embodiments, the therapeutically effective amount is from 5 GBq to 10 GBq. In some aspects of these embodiments, the therapeutically effective amount is from 6 GBq to 9 GBq. In some aspects of these embodiments, the therapeutically effective amount is from 6.5 GBq to 8.5 GBq. In some aspects of these embodiments, the therapeutically effective amount is from 7 GBq to 8 GBq. In some aspects of these embodiments, the therapeutically effective amount is 7.4 GBq.

In some aspects of these embodiments, the therapeutically effective amount is from about 0.1 mg/m² to about 6.0 mg/m². In some aspects of these embodiments, the therapeutically effective amount is from about 0.1 mg/m² to about 5.0 mg/m². In some aspects of these embodiments, the therapeutically effective amount is from about 0.1 mg/m² to about 4.0 mg/m². In some aspects of these embodiments, the therapeutically effective amount is from about 0.1 mg/m² to about 3.5 mg/m². In some aspects of these embodiments, the therapeutically effective amount is from about 0.1 mg/m² to about 3.0 mg/m². In some aspects of these embodiments, the therapeutically effective amount is from about 0.1 mg/m² to about 2.5 mg/m². In some aspects of these embodiments, the therapeutically effective amount is from about 0.1 mg/m² to about 2.0 mg/m².

In some aspects of these embodiments, the therapeutically effective amount is from 0.1 mg/m² to 6.0 mg/m². In some aspects of these embodiments, the therapeutically effective amount is from 0.1 mg/m² to 5.0 mg/m². In some aspects of these embodiments, the therapeutically effective amount is from 0.1 mg/m² to 4.0 mg/m². In some aspects of these embodiments, the therapeutically effective amount is from 0.1 mg/m² to 3.5 mg/m². In some aspects of these embodiments, the therapeutically effective amount is from 0.1 mg/m² to 3.0 mg/m². In some aspects of these embodiments, the therapeutically effective amount is from 0.1 mg/m² to 2.5 mg/m². In some aspects of these embodiments, the therapeutically effective amount is from 0.1 mg/m² to 2.0 mg/m².

In other aspects, the methods and uses described herein further comprise imaging PSMA expression by the cancer. In some aspects of these embodiments, the step of imaging occurs before the step of administering. In some aspects of these embodiments, the imaging is performed by imaging wherein the imaging is selected from the group consisting of SPECT imaging, PET imaging, IHC, and FISH. In some aspects of these embodiments, the imaging is performed by SPECT imaging.

In some aspects of these embodiments, the step of imaging comprises administering to the patient a PSMA ligand-imaging conjugate of the formula 2

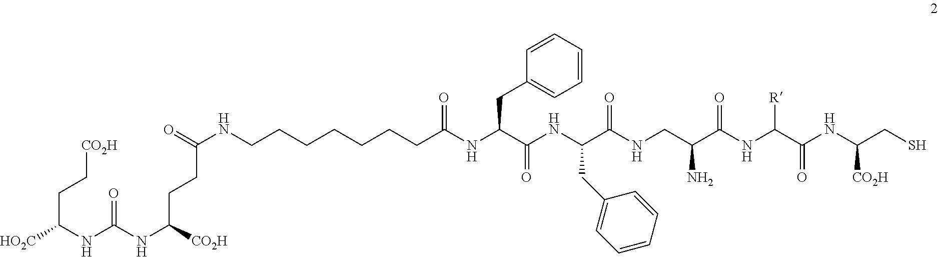

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of imaging comprises administering a PSMA ligand-imaging conjugate of the formula 3

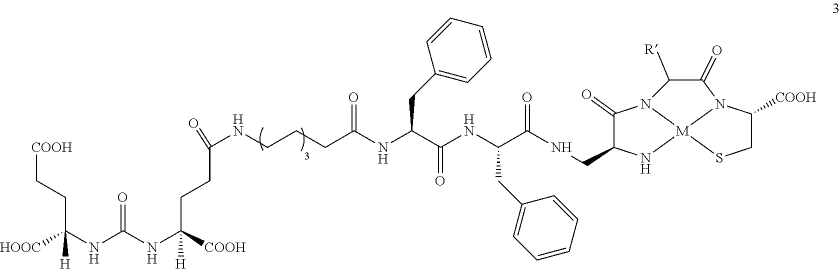

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the PSMA ligand-imaging conjugate is of the formula 2a

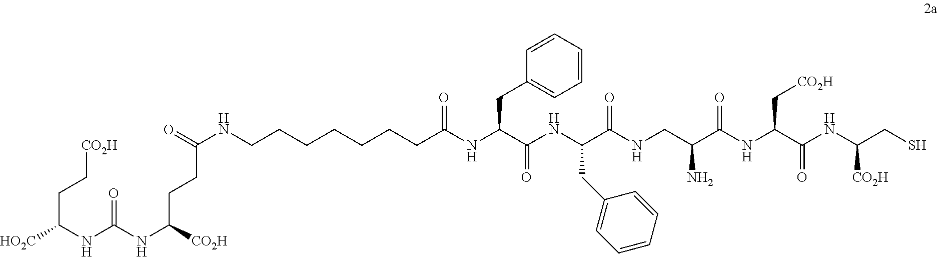 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate. In some aspects of these embodiments, the PSMA ligand-imaging conjugate is of the formula 3a

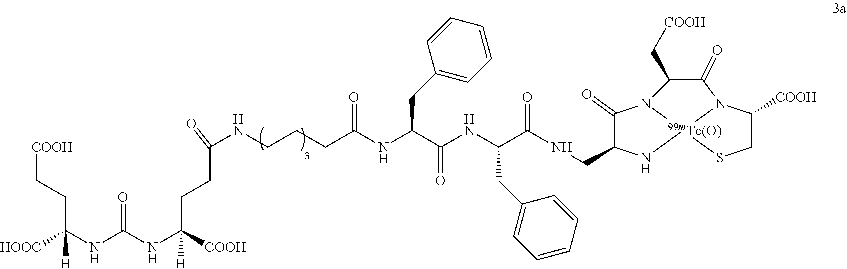

or a pharmaceutically acceptable salt thereof.

In some aspects of these embodiments, the step of imaging comprises administering to the patient a PSMA ligand-imaging conjugate of the formula 4

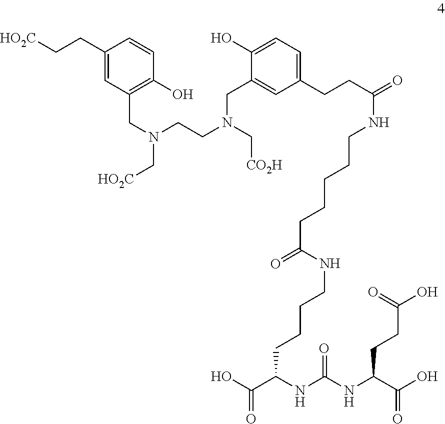 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

or a pharmaceutically acceptable salt thereof, wherein a radionuclide is bound to the conjugate. In some aspects of these embodiments, the radionuclide is ⁶⁸Ga.

In other aspects, the methods and uses described herein further comprise determining the PSMA status of the patient by imaging. In some aspects of these embodiments, the imaging is SPECT imaging. In some aspects of these embodiments, the PSMA status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease. In some aspects of these embodiments, the PSMA positive lesions indicate functionally active PSMA.

In some aspects of these embodiments, the step of determining comprises administering to the patient a PSMA ligand-imaging conjugate of the formula 2

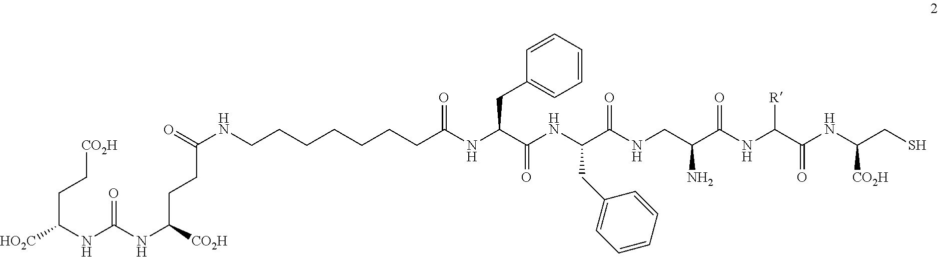

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein the conjugate is bound to a radionuclide.

In some aspects of these embodiments, the step of determining comprises administering a PSMA ligand-imaging conjugate of the formula 3

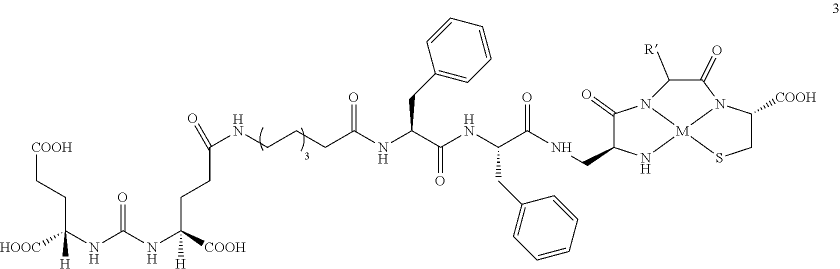

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the PSMA ligand-imaging conjugate is of the formula 2a

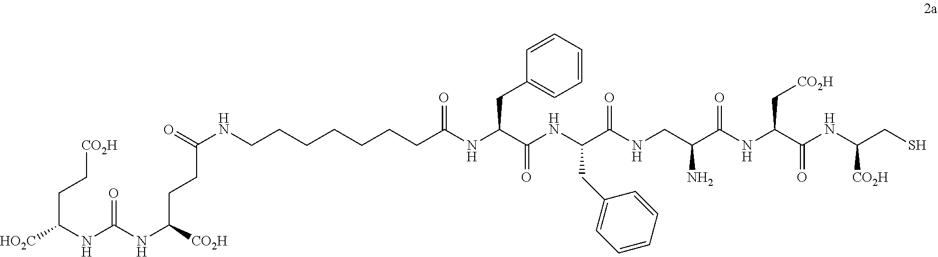 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate.

In some aspects of these embodiments, the PSMA ligand-imaging conjugate is of the formula 3a

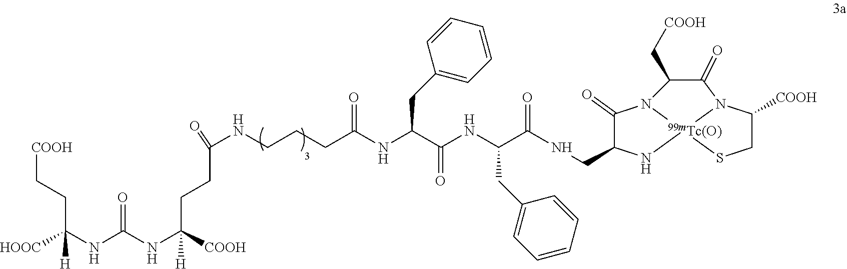

or a pharmaceutically acceptable salt thereof.

In some aspects of these embodiments, the step of determining comprises administering to the patient a PSMA ligand-imaging conjugate of the formula 4

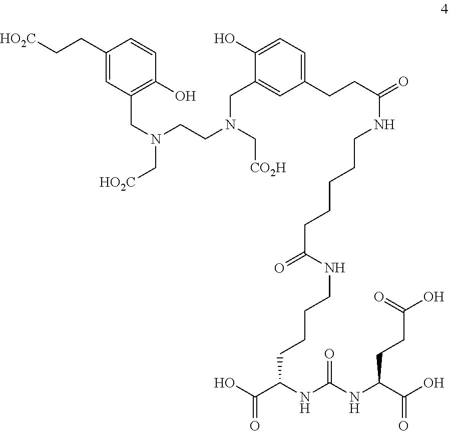 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

or a pharmaceutically acceptable salt thereof, wherein a radionuclide is bound to the conjugate. In some aspects of these embodiments, the radionuclide is ⁶⁸Ga.

In other embodiments, the present disclosure provides a method of treating a cancer in a patient in need of such treatment comprising, administering to the patient a therapeutically effective amount of a Compound 1

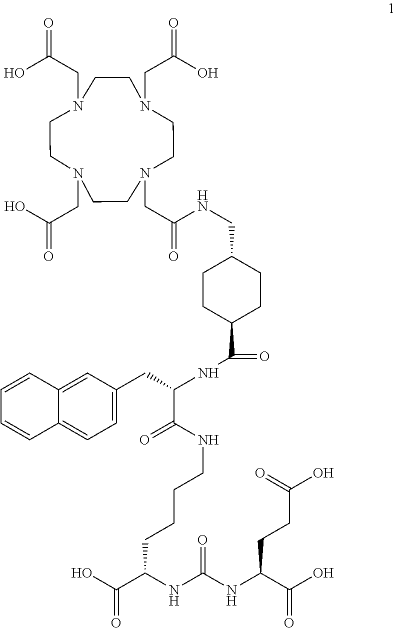 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the Compound 1, wherein stable disease results after the Compound 1, or a pharmaceutically acceptable salt thereof, is administered.

In other embodiments, the present disclosure provides use of a Compound 1

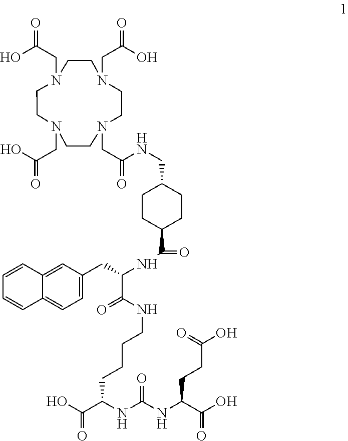 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the Compound 1, wherein stable disease results after the Compound 1, or a pharmaceutically acceptable salt thereof, is administered. In some aspects of these embodiments, the use comprises administering to the patient a therapeutically effective amount of the Compound 1.

In other embodiments, the present disclosure provides use of a Compound 1

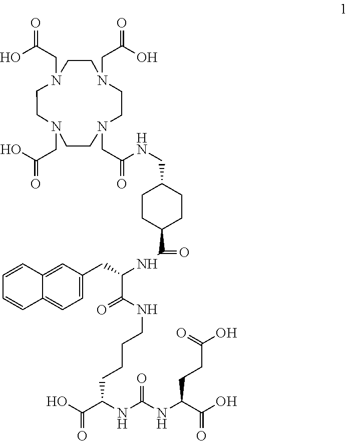 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the Compound 1, in the preparation of a medicament useful for the treatment of a cancer in a patient, wherein stable disease results after the Compound 1, or a pharmaceutically acceptable salt thereof, is administered. In some aspects, the medicament comprises a therapeutically effective amount of the Compound 1, or a pharmaceutically acceptable salt thereof.

In some aspects of these embodiments, the patient has been treated with at least one prior treatment. In some aspects of these embodiments, the at least one prior treatment is selected from the group consisting of an androgen axis systemic treatment, a chemotherapeutic agent, surgery, radiation therapy, immunotherapy, photodynamic therapy, stem cell therapy, and hyperthermia. In some aspects of these embodiments, the at least one prior treatment is a systemic treatment. In some aspects of these embodiments, the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof. In some aspects of these embodiments, the cancer is a PSMA expressing cancer. In some aspects of these embodiments, the compound is at least about 98 percent pure.

Embodiments of the invention are further described by the following enumerated clauses:
1. A method for treating a cancer in a patient in need of such treatment comprising, administering to the patient a therapeutically effective amount of a compound of the formula 1 or 2

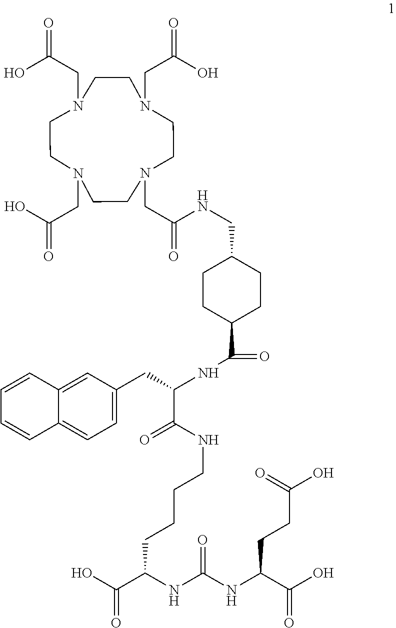 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein the compound is complexed with a metal.
2. The method of clause 1, wherein the cancer is a PSMA expressing cancer.
3. The method of clause 1 or 2, wherein the compound of the formula 1 is at least about 98 percent pure.
4. The method of any one of the preceding clauses, wherein the cancer is prostate cancer.
5. The method of any one of the preceding clauses, wherein the cancer is metastatic castration-resistant prostate cancer.
6. The method of any one of the preceding clauses, wherein the compound of the formula 1, is administered in a parenteral dosage form.
7. The method of clause 6, wherein the parenteral dosage form is selected from the group consisting of intradermal, subcutaneous, intramuscular, intraperitoneal, intravenous, and intrathecal.
8. The method of any one of the preceding clauses, wherein the therapeutically effective amount is from about 2 GBq to about 13 GBq.
9. The method of any one of the preceding clauses, wherein the therapeutically effective amount is from about 4 GBq to about 11 GBq.
10. The method of any one of the preceding clauses, wherein the therapeutically effective amount is from about 5 GBq to about 10 GBq.
11. The method of any one of the preceding clauses, wherein the therapeutically effective amount is from about 6 GBq to about 9 GBq.
12. The method of any one of the preceding clauses, wherein the therapeutically effective amount is from about 6.5 GBq to about 8.5 GBq.
13. The method of any one of the preceding clauses, wherein the therapeutically effective amount is from about 7 GBq to about 8 GBq.
14. The method of any one of the preceding clauses, wherein the therapeutically effective amount is about 7.4 GBq.
15. The method of any one of the preceding clauses, further comprising imaging PSMA expression by the cancer.
16. The method of clause 15, wherein the imaging occurs before the step of administering.
17. The method of clause 16, wherein the imaging is performed by imaging and wherein the imaging is selected from the group consisting of SPECT imaging, PET imaging, IHC, and FISH.
18. The method of clause 17, wherein the imaging is performed by SPECT imaging.
19. The method of any one of clauses 1 to 14, further comprising determining the PSMA status of the patient by imaging.
20. The method of clause 19, wherein the imaging is SPECT imaging.
21. The method of clause 20, wherein the PSMA status of the patient correlates with a clinical benefit to the patient.
22. The method of clause 21, wherein the clinical benefit is selected from the group consisting of inhibition of tumor growth, stable disease, a partial response, and a complete response.
23. The method of clause 22, wherein the clinical benefit is stable disease.
24. The method of clause 21, wherein at least one PSMA positive lesion indicates functionally active PSMA.
25. The method of any one of the preceding clauses, wherein the patient has been treated with at least one prior treatment.
26. The method of clause 25, wherein the at least one prior treatment is selected from the group consisting of an androgen axis systemic treatment, chemotherapeutic agent, surgery, radiation therapy, immunotherapy, photodynamic therapy, stem cell therapy, and hyperthermia.
27. The method of clause 26, wherein the at least one prior treatment is an androgen axis drug systemic treatment.
28. The method of clause 26, wherein the at least one prior treatment is selected from the group consisting of abiraterone, orteronel, galeterone, seviteronel, apalutamide, enzalutamide, and combinations thereof.
29. The method of clause 25, wherein the at least one prior treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.
30. The method of any one of the preceding clauses, wherein the compound of the formula 1 is administered in combination with a second treatment.
31. The method of clause 30, wherein the second treatment is best supportive treatment.
32. The method of clause 30, wherein the second treatment is best standard of care treatment.
33. The method of clause 30, wherein the second treatment is best supportive/best standard of care treatment.
34. The method of clause 30, wherein the second treatment is an androgen axis systemic treatment.
35. The method of clause 34, wherein the androgen axis systemic treatment is selected from the group consisting of abiraterone, orteronel, galeterone, seviteronel, apalutamide, enzalutamide, and combinations thereof.
36. The method of clause 30, wherein the second treatment is radiation therapy.
37. The method of clause 30, wherein the radiation therapy is external beam radiation therapy (EBRT).
38. The method of any one of the preceding clauses, wherein the compound of the formula 1 is administered on a schedule of once per week.
39. The method of any one of the clauses 1 to 37, wherein the compound of the formula 1 is administered on a schedule of once per every 2 weeks.
40. The method of any one of the clauses 1 to 37, wherein the compound of the formula 1 is administered on a schedule of once per every 3 weeks.
41. The method of any one of the clauses 1 to 37, wherein the compound of the formula 1 is administered on a schedule of once per every 4 weeks.
42. The method of any one of the clauses 1 to 37, wherein the compound of the formula 1 is administered on a schedule of once per every 5 weeks.
43. The method of any one of the clauses 1 to 37, wherein the compound of the formula 1 is administered on a schedule of once per every 6 weeks.
44. The method of any one of the clauses 1 to 37, wherein the compound of the formula 1 is administered on a schedule of once per every 7 weeks.
45. The method of any one of the clauses 1 to 37, wherein the compound of the formula 1 is administered on a schedule of once per every 8 weeks.
46. The method of any one of the clauses 1 to 37, wherein the compound of the formula 1 is administered on a schedule of once per every 4 to 6 weeks.
47. The method of any one of clauses 38 to 46, wherein the compound of the formula 1 is administered for about 2 to 8 cycles of the schedule.
48. The method of any one of clauses 38 to 46, wherein the compound of the formula 1 is administered for about 3 to 7 cycles of the schedule.
49. The method of any one of clauses 38 to 46, wherein the compound of the formula 1 is administered for about 4 to 6 cycles of the schedule.
50. The method of any one of the preceding clauses, wherein the metal complexed to the Compound 1 or the Compound 2 is selected from the group consisting of ⁹⁰Y¹⁷⁷Lu, ⁶⁴Cd, ¹⁵³Gd, ¹⁵⁵Gd, ¹⁵⁷Gd, ²¹³Bi, and ²²⁵Ac.
51. The method of clause 50, wherein the metal complexed to the Compound 1 is ¹⁷⁷Lu.
52. The method of clause 50, wherein the metal complexed to the Compound 1 is ²²⁵Ac.

### BRIEF DESCRIPTION OF THE DRAWINGS

FIG. 1 shows a schematic of the treatment method design.

### DEFINITIONS

In accordance with the invention, “functionally active PSMA” means a cell surface membrane-bound glycoprotein that binds to a PSMA ligand. It will be appreciated that PSMA ligands are well known to those skilled in the art such as those described in US patent publication no. US 2010/0324008 A1, incorporated herein by reference.

In accordance with the invention, “clinical benefit” means a response of a patient to treatment with Compound 1 where the response includes overall survival of the patient, ability to receive four or more cycles of therapy (e.g., four weeks of therapy) with Compound 1, inhibition of tumor growth, stable disease, a partial response, and/or a complete response, among other clinical benefits defined by the Food and Drug Administration in the United States of America.

In accordance with the invention, “inhibition of tumor growth” means reduction in tumor size, complete disappearance of a tumor, or growth of a patient tumor of less than 30% over the course of therapy with Compound 1.

In accordance with the invention, “stable disease” means no material progression of disease in a patient over the course of therapy with Compound 1.

In accordance with the invention, “a partial response” means a decrease in tumor size of 30% or greater in a patient treated with Compound 1.

In accordance with the invention, “a complete response” means the disappearance of detectable disease in a patient treated with Compound 1.

In accordance with the invention, “prior treatment” means the patient has been treated with at least one prior treatment known in the art. It will be appreciated that a prior treatment can be any treatment known to those of skill in the art, including, but not limited, chemotherapeutic agent, surgery, radiation therapy, immunotherapy, photodynamic therapy, stem cell therapy, hyperthermia, and the like. Prior treatments can include systemic treatments including, but not limited to treatment with abiraterone, orteronel, galeterone, seviteronel, apalutamide, enzalutamide, palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

In accordance with the inventions, the term “alkyl” includes a chain of carbon atoms, which is optionally branched. It will be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. Illustrative alkyl groups include, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. As used herein, a “carboxyalkyl” group includes a combination of an “alkyl” group as described herein with a “carboxy” group. As used herein, a “hydroxyalkyl” group includes a combination of an “alkyl” group as described herein with a “hydroxy” group. As used herein, a “aminoalkyl” group includes a combination of an “alkyl” group as described herein with a “amino” group.

In accordance with the invention, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium.

In accordance with the invention, the term “aryl” includes monocyclic and polycyclic aromatic carbocyclic groups having from 6 to 14 ring carbon atoms, each of which may be optionally substituted. Illustrative aromatic carbocyclic groups described herein include, but are not limited to, phenyl, naphthyl, and the like. In accordance with the invention, the term “heteroaryl” includes aromatic heterocyclic groups, having from 5 to 10 ring atoms, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like. In accordance with the invention, the term “heteroarylalkyl” includes a combination of an “alkyl” group as described herein with a “heteroaryl” group described herein. In accordance with the invention, the term “arylalkyl” includes a combination of an “alkyl” group as described herein with a “aryl” group described herein, for example a benzyl group.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

In accordance with the invention, the term “administering” as used herein includes all means of introducing the Compound 1 and PSMA ligand-imaging conjugates described herein to the patient, including, but not limited to, oral (po), intravenous (iv), intramuscular (im), subcutaneous (sc), transdermal, inhalation, buccal, ocular, sublingual, vaginal, rectal, and the like. The Compound 1 and PSMA ligand-imaging conjugates described herein may be administered in unit dosage forms and/or formulations containing conventional nontoxic pharmaceutically-acceptable carriers, adjuvants, and vehicles.

In accordance with the invention, “becquerel” means a SI derived unit of radioactivity as it is commonly understood by one of skill in the art. One becquerel is defined as the activity of a quantity of radioactive material in which one nucleus decays per second. A becquerel is therefore equivalent to an inverse second, s−1. The becquerel is known to one of skill in the art as the successor of the curie (Ci), an older, non-SI unit of radioactivity based on the activity of 1 gram of radium-226. The curie is defined as 3.7. 1010 s−1, or 37 GBq.

In accordance with the invention, “curie” or “Ci” means a unit of radioactivity named after the French physicist and chemist Marie Curie as commonly understood by one of skill in the art. The prefixes milli and micro are from the metric system and represent 0.001 and 0.000001, respectively. So, a millicurie (mCi) is 0.001 curie. A microcurie (μCi) is 0.000001 curie.

### DETAILED DESCRIPTION

In accordance with Applicant's invention described herein, the embodiments of the numbered clauses provided in the summary above, or any combination thereof, are contemplated for combination with any of the embodiments described in the Detailed Description section of this patent application.

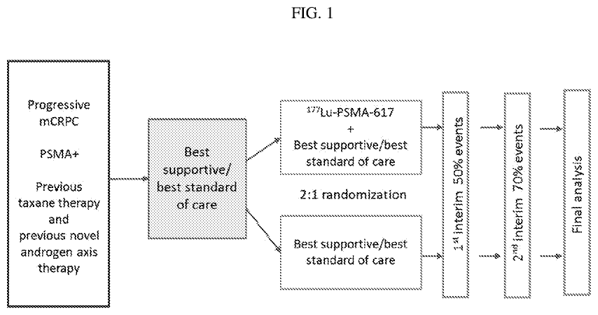

Referring to FIG. 1, the method design can be described according to the schematic shown. In some embodiments, stratification factors for the design include, but are not limited to serum lactate dehydrogenase (LDH) (</=260 IU/L v. >260 IU/L), presence of liver metastases, ECOG score (0-1 v. 2), inclusion of NAAD in best supportive/best standard of care, and the like. In some embodiments, the primary endpoint can be overall survival. In some embodiments, secondary endpoints include, but are not limited to, radiographic progression-free survival (rPFS), RECIST response, time to first symptomatic skeletal event (SSE), and the like. In some embodiments, additional secondary endpoints include, but are not limited to, safety and tolerability, heather-related quality of life (HRQoL; EQ-5D-5L, FACT-P and Brief Pain Inventory—Short FORM [BPI-SF]), health economics, progression-free survival (PFS) (radiological, clinical or PSA progression), biochemical response, such as PSA levels, alkaline phosphatase level, and/or lactate dehydrogenase level. In some embodiments, an endpoint for the treatment methods described herein can be a patient who has achieved a >1=50% decrease from baseline that is confirmed by a second PSA measurement >/=4 weeks. In some embodiments, an endpoint for the treatment methods described herein can be a patient who has achieved a >/=40% decrease from baseline that is confirmed by a second PSA measurement >/=4 weeks. In some embodiments, an endpoint for the treatment methods described herein can be a patient who has achieved a >/=30% decrease from baseline that is confirmed by a second PSA measurement >/=4 weeks.

In one embodiment, the methods described herein can be used for both human clinical medicine and veterinary applications. Thus, a “patient” can be administered the Compound 1 or PSMA ligand-imaging conjugates described herein, and can be human or, in the case of veterinary applications, can be a laboratory, agricultural, domestic, or wild animal. In one aspect, the patient can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In some embodiments, patients with PSMA positive scans can be randomized in a 2:1 ratio to receive either Compound 1 plus best supportive/best standard of care or to receive best supportive/best standard of care only. In some embodiments, best supportive/best standard of care can be determined by the treating physician/investigator. In some embodiments, best supportive/best standard of care can be determined by the treating physician/investigator, but will exclude investigational agents, cytotoxic chemotherapy, other systemic radioisotopes, and hemi-body radiotherapy. In some embodiments, novel androgen axis drugs [NAADs], such as abiraterone or enzalutamide, are allowed.

In some embodiments, patients will be monitored throughout the 6 to 10-month treatment period for survival, disease progression, and adverse events. In some embodiments, a long-term follow-up period can include the collection of survival and treatment updates, adverse events assessment, as well as blood for hematology and chemistry testing.

In some embodiments, the patient is 18 Years of age or older. In some embodiments, the patient is a male. In some embodiments, the patient has previously been diagnosed with prostate cancer. In some embodiments, the patient has been previously diagnosed with metastatic castration-resistant prostate cancer (mCRPC). In some embodiments, the patient meets one or more criteria, selected from the group consisting of Eastern Cooperative Oncology Group (ECOG) performance status of 0 to 2; a life expectancy at least 6 months; histological, pathological, and/or cytological confirmation of prostate cancer; a positive ⁶⁸Ga-PSMA-11 PET/CT scan; prior orchiectomy and/or ongoing androgen deprivation therapy and a castrate level of serum testosterone (<50 ng/dL or <1.7 nmol/L); previously received at least one NAAD, such as enzalutamide and/or abiraterone; previously treated with at least 1 or 2 previous taxane regimens, wherein a taxane regimen comprises a minimum exposure of 2 cycles of a taxane, or previously received only one taxane regimen, and a. the patient is not willing to receive a second taxane regimen, or b. The patient's physician deems him unsuitable to receive a second taxane regimen, such as due to frailty assessed by geriatric or health status evaluation or intolerance; progressive mCRPC, such as documented progressive mCRPC based on at least one criteria, such as a. serum PSA progression defined as 2 consecutive increases in PSA over a previous reference value measured at least 1 week prior, where the minimal start value is 2.0 ng/mL, b. soft-tissue progression defined as an increase >20% in the sum of the diameter (SOD) (short axis for nodal lesions and long axis for non-nodal lesions) of all target lesions based on the smallest SOD since treatment started or the appearance of one or more new lesions, and c. progression of bone disease, such as evaluable disease or new bone lesions(s) by bone scan (2+2 PCWG3 criteria); at least one metastatic lesion that is present on baseline CT, MRI, or bone scan imaging obtained ≤28 days prior to beginning therapy with Compound 1; recovered to <Grade 2 from all clinically significant toxicities related to prior therapies, such as prior chemotherapy, radiation, immunotherapy, and the like; adequate organ function, such as a. bone marrow reserve including white blood cell (WBC) count ≥2.5×109/L (2.5×10\hat{ }9/L is equivalent to 2.5×10³/μL and 2.5×K/μL and 2.5×10³/cumm and 2500/μL) or absolute neutrophil count (ANC)≥1.5×10⁹/L (1.5×10⁹/L is equivalent to 1.5×10³/μL and 1.5×K/μL and 1.5×10³/cumm and 1500/μL), platelets≥100×10\hat{ }9/L (100×10\hat{ }9/L is equivalent to 100×10\hat{ }3/μL and 100×K/μL and 100×10\hat{ }3/cumm and 100,000/μL), and/or hemoglobin≥9 g/dL (9 g/dL is equivalent to 90 g/L and 5.59 mmol/L); b. hepatic, such as total bilirubin ≤1.5× the institutional upper limit of normal (ULN) (for patients with known Gilbert's Syndrome ≤3×ULN is permitted), alanine aminotransferase (ALT) or aspartate aminotransferase (AST)≤3.0×ULN OR≤5.0×ULN for patients with liver metastases, and c. renal, such as serum creatinine ≤1.5×ULN or creatinine clearance ≥50 m/min; albumin >3.0 g/dL (3.0 g/dL is equivalent to 30 g/L); and a stable bisphosphonate or denosumab regimen for ≥30 days prior to treatment.

In some embodiments, a patient may not receive treatment if the patient has one of more of previous treatment with Strontium-89, Samarium-153, Rhenium-186, Rhenium-188, Radium-223 or hemi-body irradiation within about 6 months prior treatment; previous PSMA-targeted radioligand therapy; previous systemic anti-cancer therapy (e.g. chemotherapy, immunotherapy or biological therapy [including monoclonal antibodies]) within about 28 days prior to treatment; previous administration of investigational agents within about 28 days prior to treatment; a known hypersensitivity to the components of the therapy or its analogs; any other concurrent cytotoxic chemotherapy, immunotherapy, radioligand therapy, or investigational therapy; a transfusion within about 30 days of treatment; a history of CNS metastases that have received therapy (surgery, radiotherapy, gamma knife) and are neurologically stable, asymptomatic, and not receiving corticosteroids for the purposes of maintaining neurologic integrity; a superscan as seen in the baseline bone scan; a symptomatic cord compression, or clinical or radiologic findings indicative of impending cord compression; concurrent serious (as determined by a physician) medical conditions, including, but not limited to, New York Heart Association class III or IV congestive heart failure, history of congenital prolonged QT syndrome, uncontrolled infection, active hepatitis B or C, or other significant co-morbid conditions that in the opinion of the investigator would impair treatment or cooperation; or been diagnosed with other malignancies that are expected to alter life expectancy or may interfere with disease assessment.

In various embodiments, the cancers described herein can be a cancer cell population that is tumorigenic, including benign tumors and malignant tumors, or the cancer can be non-tumorigenic. The cancer can arise spontaneously or by such processes as mutations present in the germline of the patient or somatic mutations, or the cancer can be chemically-, virally-, or radiation-induced. Cancers applicable to the invention described herein include, but are not limited to, a glioma, a carcinoma, a sarcoma, a lymphoma, a melanoma, a mesothelioma, a nasopharyngeal carcinoma, a leukemia, an adenocarcinoma, and a myeloma.

In some aspects the cancers can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction.

### Compound 1 has the Formula

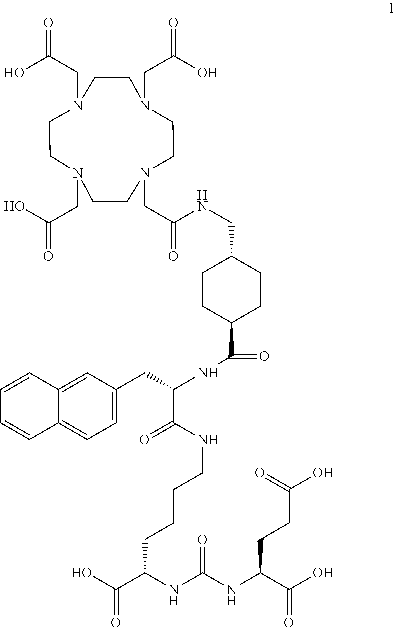 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the compound.

In other embodiments, any of a variety of PSMA ligand-imaging conjugates detectable by PET imaging, SPECT imaging, and the like can be used. The exact manner of imaging is not limited to the imaging agents described herein. Collectively, the PSMA ligand-imaging conjugates useful for imaging described herein, including those described by formulas and the agents useful for PET imaging, SPECT imaging, etc. are referred to as “PSMA ligand-imaging conjugates.”

In one embodiment, the Compound 1 and PSMA ligand-imaging conjugates described herein bind to expressed PSMA on cancer cells. In one illustrative aspect, the Compound 1 and PSMA ligand-imaging conjugates are capable of differentially binding to PSMA on cancer cells compared to normal cells due to preferential expression (or over-expression) of PSMA on the cancer cells.

In other embodiments of the methods described herein, pharmaceutically acceptable salts of the Compound 1 and PSMA ligand-imaging conjugates described herein are provided. Pharmaceutically acceptable salts of the Compound 1 and PSMA ligand-imaging conjugates described herein include acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the Compound 1 and PSMA ligand-imaging conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the Compound 1 and PSMA ligand-imaging conjugates described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of Compound 1 and PSMA ligand-imaging conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of injectable compositions can be brought about by including in the composition an agent which delays absorption, for example, monostearate salts and gelatin.

Illustrative formats for oral administration include tablets, capsules, elixirs, syrups, and the like.

Depending upon the cancer type as described herein, the route of administration and/or whether the Compound 1 and/or PSMA ligand-imaging conjugates are administered locally or systemically, a wide range of permissible dosages are contemplated herein, including doses falling in the range from about 1 μg/kg to about 1 g/kg. In some embodiments, permissible dosages are contemplated herein in the units GBq, including doses falling in the range from about 2 GBq to about 13 GBq. The dosages may be single or divided, and may administered according to a wide variety of protocols, including q.d., b.i.d., t.i.d., or even every other day, biweekly (b.i.w.), once a week, once a month, once a quarter, and the like. In each of these cases it is understood that the therapeutically effective amounts described herein correspond to the instance of administration, or alternatively to the total daily, weekly, monthly, or quarterly dose, as determined by the dosing protocol. In some embodiments, the compound of the formula 1 can be administered once per week, or once every two weeks, or once every three weeks, or once every four weeks, or once every five weeks, or once every six weeks, or once every seven weeks, or once every eight weeks, and the like

In one aspect, a Compound 1 or a PSMA ligand-imaging conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the Compound 1 or PSMA ligand-imaging conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a Compound 1 or a PSMA ligand-imaging conjugate used in the preparation of a parenteral formulation may be increased by the use of appropriate formulation techniques, such as the incorporation of solubility-enhancing agents.

In various embodiments, formulations for parenteral administration may be formulated for immediate and/or modified release. In one illustrative aspect, active agents of the invention (i.e., the Compound 1 or PSMA ligand-imaging conjugates) may be administered in a time release formulation, for example in a composition which includes a slow release polymer. The active Compound 1 or PSMA ligand-imaging conjugates can be prepared with carriers that will protect the Compound 1 or PSMA ligand-imaging conjugate against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the Compound 1 or PSMA ligand-imaging conjugates described herein or compositions comprising the Compound 1 or PSMA ligand-imaging conjugates may be continuously administered, where appropriate.

In one embodiment, a kit is provided. If a combination of active Compound 1 and PSMA ligand-imaging conjugates is to be administered, two or more pharmaceutical compositions may be combined in the form of a kit suitable for sequential administration or co-administration of the compositions. Such a kit comprises two or more separate pharmaceutical compositions, at least one of which contains a Compound 1 or PSMA ligand-imaging conjugate described herein, and means for separately retaining the compositions, such as a container, divided bottle, or divided foil packet. In another embodiment, compositions comprising one or more of the Compound 1 or PSMA ligand-imaging conjugates described herein, in containers having labels that provide instructions for use of the Compound 1 or PSMA ligand-imaging conjugates for patient selection and/or treatment are provided.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active Compound 1 or PSMA ligand-imaging conjugate into a sterile vehicle which contains a dispersion medium and any additional ingredients of those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering Compound 1 can be used. For example, Compound 1 can be administered as single doses, or the doses can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of Compound 1 to treat the cancer. In one embodiment, the patient is injected multiple times (preferably about 2 up to about 50 times) with Compound 1, for example, at 12-72 hour intervals or at 48-72 hour intervals. Additional injections of Compound 1 can be administered to the patient at an interval of days or months after the initial injections(s) and the additional injections can prevent recurrence of the cancer.

Any suitable course of therapy with Compound 1 can be used. In one embodiment, individual doses and dosage regimens are selected to provide a total dose administered during a month of about 15 mg. In one illustrative example, Compound 1 is administered in a single daily dose administered five days a week, in weeks 1, 2, and 3 of each 4 week cycle, with no dose administered in week 4. In an alternative example, Compound 1 is administered in a single daily dose administered three days a week, of weeks 1, and 3 of each 4 week cycle, with no dose administered in weeks 2 and 4. In an alternative example, Compound 1 is administered biweekly on weeks 1 and 2, i.e. on days 1, 4, 8, 11, of a 3-week cycle. In an alternative example, Compound 1 is administered once weekly on weeks 1 and 2, i.e. days 1 and 8 of a 3-week cycle.

The unitary daily dosage of the Compound 1 can vary significantly depending on the patient condition, the cancer being treated, the route of administration of the Compound 1 and tissue distribution, and the possibility of co-usage of other therapeutic treatments, such as radiation therapy or additional drugs in combination therapies. The effective amount to be administered to a patient is based on body surface area, mass, and physician assessment of patient condition. Therapeutically effective doses (also referred to herein as “therapeutically effective amount”) can range, for example, from about 0.5 mg/m² to about 10.0 mg/m². The therapeutically effective doses described herein also include ranges of about 0.5 mg/m² to about 9.5 mg/m², about 0.5 mg/m² to about 9.0 mg/m², about 0.5 mg/m² to about 8.5 mg/m², about 0.5 mg/m² to about 8.0 mg/m², about 0.5 mg/m² to about 7.5 mg/m², about 0.5 mg/m² to about 7.0 mg/m², about 0.5 mg/m² to about 6.5 mg/m², about 0.5 mg/m² to about 6.0 mg/m², about 0.5 mg/m² to about 5.5 mg/m², about 0.5 mg/m² to about 5.0 mg/m², about 0.5 mg/m² to about 4.5 mg/m², about 0.5 mg/m² to about 4.0 mg/m², about 0.5 mg/m² to about 3.5 mg/m², about 0.5 mg/m² to about 3.0 mg/m², about 0.5 mg/m² to about 2.5 mg/m², about 0.5 mg/m² to about 2.0 mg/m², about 0.5 mg/m² to about 1.5 mg/m², about 1.0 mg/m² to about 9.5 mg/m², about 1.0 mg/m² to about 9.0 mg/m², about 1.0 mg/m² to about 8.5 mg/m², about 1.0 mg/m² to about 8.0 mg/m², about 1.0 mg/m² to about 7.5 mg/m², about 1.0 mg/m² to about 7.0 mg/m², about 1.0 mg/m² to about 6.5 mg/m², about 1.0 mg/m² to about 6.0 mg/m², about 1.0 mg/m² to about 5.5 mg/m², about 1.0 mg/m² to about 5.0 mg/m², about 1.0 mg/m² to about 4.5 mg/m², about 1.0 mg/m² to about 4.0 mg/m², about 1.0 mg/m² to about 3.5 mg/m², about 1.0 mg/m² to about 3.0 mg/m², about 1.0 mg/m² to about 2.5 mg/m², about 1.0 mg/m² to about 2.0 mg/m², and about 1.0 mg/m² to about 1.5 mg/m². One of skill in the art will readily appreciate that the therapeutically effective dose may vary within the various ranges provided above based on the factors noted above. The therapeutically effective dose for any particular patient or group of patients may be any number value between about 0.5 mg/m² and about 10.0 mg/m², including but not limited to 1.0 mg/m², 1.5, mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m², 7.0 mg/m², 7.5 mg/m², 8.0 mg/m², 8.5 mg/m², 9.0 mg/m², 9.5 mg/m² and 10.0 mg/m². The total dose may be administered in single or divided doses and may, at the physician's discretion, fall outside of the typical range given herein.

In some embodiments, the compound of the formula 1 can administered in combination with a second treatment. In some embodiments, the second treatment is best supportive treatment. In some embodiments, the second treatment is best standard of care treatment. In some embodiments, the second treatment is best supportive/best standard of care treatment. In some embodiments, the second treatment is an androgen axis systemic treatment. In some embodiments, the androgen axis systemic treatment is selected from the group consisting of abiraterone, orteronel, galeterone, seviteronel, apalutamide, enzalutamide, and combinations thereof. In some embodiments, the second treatment is radiation therapy. In some embodiments, the radiation therapy is external beam radiation therapy (EBRT).

The PSMA ligand-imaging conjugates and Compound 1 described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The PSMA ligand-imaging conjugates and Compound 1 described herein may be capable of existing as geometric isomers. Accordingly, it is to be understood that the present invention includes pure geometric isomers or mixtures of geometric isomers.

It is appreciated that the PSMA ligand-imaging conjugates and Compound 1 described herein may exist in unsolvated forms as well as solvated forms, including hydrated forms. In general, the solvated forms are equivalent to unsolvated forms and are encompassed within the scope of the present invention. The PSMA ligand-imaging conjugates and Compound 1 described herein may exist in multiple crystalline or amorphous forms. In general, all physical forms are equivalent for the uses contemplated by the present invention and are intended to be within the scope of the present invention.

In another embodiment, compositions and/or dosage forms for administration of Compound 1 are prepared from Compound 1 with a purity of at least about 90%, or about 95%, or about 96%, or about 97%, or about 98%, or about 99%, or about 99.5%. In another embodiment, compositions and or dosage forms for administration of Compound 1 are prepared from Compound 1 with a purity of at least 90%, or at least 95%, or at least 96%, or at least 97%, or at least 98%, or at least 99%, or at least 99.5%.

In another embodiment, compositions and/or dosage forms for administration of the PSMA ligand-imaging conjugate are prepared from the PSMA ligand-imaging conjugate with a purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 97%, or at least 98%, or at least 99%, or at least 99.5%.

In another embodiment, compositions and/or dosage forms for administration of radiolabeled PSMA ligand-imaging conjugate are prepared from the PSMA ligand-imaging conjugate with a radiochemical purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 96%, or at least 97%, or at least 98%, or at least 99%, or at least 99.5%.

The purity of Compound 1 or the PSMA ligand-imaging conjugates described herein may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In another embodiment, the Compound 1 or PSMA ligand-imaging conjugate described herein is provided in a sterile container or package.

In one aspect, a clinical benefit of the patient to treatment with Compound 1 can be characterized as overall survival (OS). As used herein, the term “overall survival (OS)” means the time from the date of randomization to the date of death from any cause.

In one aspect, a clinical benefit of the patient to treatment with Compound 1 can be characterized utilizing Response Evaluation Criteria in Solid Tumors (RECIST) criteria. Illustratively, the criteria have been adapted from the original WHO Handbook (3), taking into account the measurement of the longest diameter for all target lesions: complete response, (CR)—the disappearance of all target lesions; partial response (PR)—at least a 30% decrease in the sum of the longest diameter of target lesions, taking as reference the baseline sum longest diameter; stable disease (SD)—neither sufficient shrinkage to qualify for partial response nor sufficient increase to qualify for progressive disease, taking as reference the smallest sum longest diameter since the treatment started; progressive disease (PD)—at least a 20% increase in the sum of the longest diameter of target lesions, taking as reference the smallest sum longest diameter recorded since the treatment started or the appearance of one or more new lesions. In another aspect overall disease response rate (ORR) is a clinical benefit and is calculated as the percent of patients who achieve a best response of CR or PR. Overall disease control rate (DCR) can be another clinical benefit and is calculated as the percent of patients who achieve a best response of CR, PR, or SD. In some embodiments, the response can be disease control rate (DCR) as measured by RECIST v1.1 criteria.

In another aspect, a clinical benefit of the patient to treatment with Compound 1 can be characterized as radiographic progression-free survival (rPFS). As used herein, “radiographic progression-free survival (rPFS)” means the time from the date of randomization to the date of radiographic disease progression as outlined in Prostate Cancer Working Group 3 (PCWG3) Guidelines or death from any cause. See, for example, Scher H I, Morris M J, Stadler W M, Higano C, Basch E, Fizazi K, et al. Trial Design and Objectives for Castration-Resistant Prostate Cancer: Updated Recommendations from the Prostate Cancer Clinical Trials Work Group 3. J Clin Oncol 2016; 34(12):1402-18. In another aspect, a clinical benefit of the patient to treatment with Compound 1 can be characterized as time to a first symptomatic skeletal event (SSE). It will be appreciated that symptomatic skeletal event means a clinically significant pathological fracture, surgery or radiation to bone, or spinal cord compression. As used herein, “time to a first symptomatic skeletal event” means date of randomization to the date of first new symptomatic pathological bone fracture, spinal cord compression, tumor-related orthopedic surgical intervention, or requirement for radiation therapy to relieve bone pain, whichever occurs first.

In one illustrative example overall survival is the time to death for a given patient defined as the number of days from the first day the patient received protocol treatment (CID1) to the date of the patient's death. All events of death can be included, regardless of whether the event occurred while the patient was still taking the study drug or after the patient discontinued the study drug. If a patient has not died, then the data can be censored at the last study visit, or the last contact date, or the date the patient was last known to be alive, whichever is last.

Alternatively, a clinical benefit of the patient as a result of treatment with Compound 1 can be characterized as inhibition of tumor growth which can be identified in a patient through, for example, follow-up imaging of the patient's cancer after treatment with Compound 1. For example, inhibition of tumor growth can be characterized by measuring the size of tumors in a patient after administration of Compound 1 according to any of the imaging techniques described herein, where the inhibition of tumor growth is indicated by a stable tumor size, or by a reduction in tumor size. It will be appreciated that the identification of inhibition of tumor growth can be accomplished using a variety of techniques, and is not limited to the imaging methods described herein (e.g CT, MRI, PET imaging, SPECT imaging or chest x-ray).

In one embodiment, a method is provided of determining whether Compound 1 is indicated for the treatment of a patient with cancer, the method comprising the step of determining the PSMA status in a patient with cancer wherein Compound 1 is indicated for the treatment of the patient if the PSMA status of the patient is positive.

In one embodiment, a method is provided of assessing whether Compound 1 is indicated for the treatment of a patient with one of the cancers described herein. The method comprises the steps of visually determining PSMA status in the patient wherein PSMA status is based on a imaging tumors that are PSMA positive in the patient, and wherein the Compound 1 is indicated for the treatment of the patient when the PSMA status of the patient is positive.

In the above-described embodiments, if a patient is in the group with positive PSMA status, a clinical benefit of Compound 1 treatment is indicated. In one embodiment, the clinical benefit to the patient can be overall survival of the patient, ability to receive four or more cycles of therapy with Compound 1, inhibition of tumor growth, stable disease, a partial response of the patient to therapy, a complete response of the patient to therapy, disease control (i.e., the best result obtained is a complete response, a partial response, or stable disease), and/or overall disease response (i.e., the best result obtained is a complete response or a partial response). In one illustrative example, the clinical benefit for a patient being treated for pleural mesothelioma or adenocarcinoma (e.g. adenocarcinoma of the gastroesophageal junction) is stable disease.

In another embodiment, the methods described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### Example 1

### A. Design:

Patients with PSMA positive scans were randomized in a 2:1 ratio to receive either Compound 1 plus best supportive/best standard of care or to receive best supportive/best standard of care only. Best supportive/best standard of care was determined by the treating physician/investigator. The study is open-label and patients are monitored throughout the 6 to 10-month treatment period for survival, disease progression, and adverse events. A long-term follow-up period includes the collection of survival and treatment updates, adverse events assessment, as well as blood for hematology and chemistry testing. During follow-up, patients are contacted every 3 months (±1 month) via phone, email, or letter for 24 months or until the overall censoring rate for survival reduces to a level identified in the SAP.

### B. Arm 1: Compound 1 Plus Best Supportive/Best Standard of Care (BS/BSOC)

Approximately 160 patients were randomized to receive the investigational product at a dose of 7.4 GBq (±10%) Compound 1 (dose is equivalent to 200 mCi) intravenously every 6 weeks (±1 week) for a maximum of 6 cycles, plus best supportive/best standard of care (BS/BSOC). After 4 cycles, patients are assessed for (1) evidence of response, (2) residual disease, and (3) tolerance to Compound 1. A saline flush with =10 mL of normal saline is administered to ensure patency of the intravenous line before administering with ¹⁷⁷Lu-PSMA-617 administration. ¹¹⁷Lu-PSMA-617 was administered slowly by the intravenous route through an indwelling catheter and followed by a saline flush. The time of administration must be recorded. The total activity administered must be measured (GBq). To-date patients have received between 1-6 cycles in the randomized arm. To-date approximately 320 patients have been scanned with Ga-PSMA-imaging Conjugate 4.

### C. Arm 2: Best Supportive/Best Standard of Care (BS/BSOC) Alone

Patients randomized to this are will receive best supportive/best standard of care (BS/BSOC) as determined by the investigator

D. Outcome measures:

Overall survival (OS) in patients with progressive PSMA-positive mCRPC who receive Compound 1 in addition to best supportive/standard of care.

1-37. (canceled)

38. A method for treating metastatic castration-resistant prostate cancer in a patient in need of such treatment, comprising administering to the patient a therapeutically effective amount of a compound of the formula 1 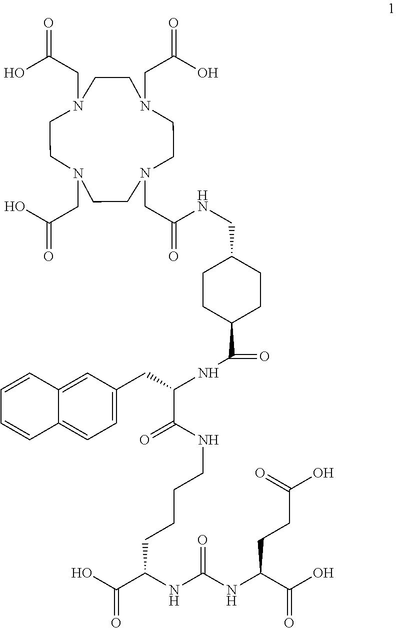 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]
wherein the compound of formula 1 is complexed with ¹⁷⁷Lu,
wherein the patient has previously been treated with at least one first androgen axis drug systemic treatment,
wherein the therapeutically effective amount of ¹⁷⁷Lu is about 7.4 GBq, and
wherein the compound of formula 1 is administered every 6 weeks (±1 week) for 6 doses.

39. The method of claim 38, wherein the patient has been treated with at least one previous taxane regimen.

40. The method of claim 39, wherein the patient has been treated with one previous taxane regimen.

41. The method of claim 39, wherein the patient has been treated with two previous taxane regimens.

42. The method of claim 38, wherein the patient has been treated with a previous chemotherapeutic agent.

43. The method of claim 42, wherein the patient has been treated with a previous administration of paclitaxel, docetaxel, or a combination thereof.

44. The method of claim 38, wherein the patient has not been treated with a previous chemotherapeutic agent.

45. The method of claim 38, wherein the patient has improved radiographic profession-free survival (rPFS).

46. The method of claim 38, wherein the first androgen axis drug systemic treatment is selected from the group consisting of abiraterone, orteronel, galeterone, seviteronel, apalutamide, enzalutamide, and combinations thereof.

47. The method of claim 38, wherein the first androgen axis drug systemic treatment is abiraterone.

48. The method of claim 38, wherein the first androgen axis drug systemic treatment is enzualutamide.

49. The method of claim 38, wherein the first androgen axis drug systemic treatment is apalutamide.

50. The method of claim 38, wherein the compound of formula 1 is administered in combination with a second androgen axis drug systemic treatment,?

51. The method of claim 50, wherein the second androgen axis drug systemic treatment is selected from the group consisting of abiraterone, orteronel, galeterone, seviteronel, apalutamide, enzalutamide, and combinations thereof.

52. The method of claim 38, wherein the compound of the formula 1 is at least about 98 percent pure.

53. The method of claim 38, wherein the compound of the formula 1, is administered in a parenteral dosage form.

54. The method of claim 53, wherein the parenteral dosage form is administered intravenously.

55. The method of claim 38, further comprising imaging PSMA expression by the cancer.

56. The method of claim 55, wherein the imaging occurs before the step of administering.

57. The method of claim 55, wherein the imaging is performed by imaging and wherein the imaging is selected from the group consisting of SPECT imaging, PET imaging, IHC, and FISH.
